(19) United States
(12) Patent Application Publication
(10) Pub. No.: US 2024/0099954 A1
(43) Pub. Date: Mar. 28, 2024

(54) HEMP PEPTIDE COMPOSITIONS FOR NUTRACEUTICAL AND PERSONAL CARE PRODUCTS

(71) Applicant: EC LABS INC., Edmonton (CA)

(72) Inventor: Louis HEERZE, Edmonton (CA)

(21) Appl. No.: 17/767,627

(22) PCT Filed: Mar. 23, 2020
(86) PCT No.: PCT/CA2020/000032

§ 371 Date: Apr. 8, 2022

Related U.S. Application Data
(60) Provisional application No. 62/928,851, filed on Oct. 31, 2019.

(51) Int. Cl. A61K 8/64; A61Q 5/00; A61Q 19/00; C07K 1/34

(52) U.S. Cl. CPC A61K 8/645; A61Q 5/00; A61Q 19/00; C07K 1/34

(57) ABSTRACT

A skin and/or hair care composition includes a mixture of low-molecular weight hemp (LMWH) peptides, some or all of the peptides are less than about 1 kDa in size. The composition may be a nutraceutical, food product, beverage or topical formulation. The peptides may be obtained by enzymatic hydrolysis of hemp proteins. The composition may be used to repair, mitigate or improve skin or hair health or conditions.

### FIELD OF THE INVENTION

The present invention relates to peptides prepared from hemp protein, compositions comprising such hemp peptides, including nutraceutical and personal care formulations, and methods of using such compositions.

### BACKGROUND

Many consumer personal care products are directed to improving the health and/or physical appearance of the skin. Among these skin care products, many are directed to delaying, minimizing or even eliminating skin wrinkling and other histological changes typically associated with the aging of skin or environmental damage to skin. Numerous compounds have been described in the art as being useful for regulating skin condition, including regulating fine lines, wrinkles and other forms of uneven or rough surface texture associated with aged or photo-damaged skin.

Skin is subject to extrinsic and intrinsic factors which may cause damage. Extrinsic factors include ultraviolet radiation, blue light emitted from electronic devices, environmental pollution, wind, heat, low humidity, harsh surfactants, abrasives, and the like. Intrinsic factors include chronological aging and other biochemical changes from within the skin. Whether extrinsic or intrinsic, these factors result in visible signs of skin aging and environmental damage, such as wrinkling and other forms of roughness (including increased pore size, flaking and skin lines), and other histological changes associated with skin aging or damage. Treatments range from cosmetic creams and moisturizers to various forms of cosmetic surgery.

Extrinsic or intrinsic factors may result in the thinning and general degradation of the skin. For example, as the skin naturally ages, there is a reduction in the cells and blood vessels that supply the skin. There is also a flattening of the dermal-epidermal junction which results in weaker mechanical resistance of this junction.

A large number of skin care actives are known in the art and used to improve the health and/or physical appearance of the skin. For example, salicylic acid and benzoyl peroxide are used in skin care compositions to treat acne. Retinoids are another example of skin care actives used in skin care compositions to reduce signs of aging skin. Although formulating skin care compositions with such actives provide skin care benefits, there are also challenges in formulating such compositions. For example, retinoid compositions typically have to be prepared under specialized conditions, such as in an inert atmosphere, and may exhibit less than optimal stability, such as discoloration, at times. Some skin care compositions may result in skin irritation, such as stinging, burning, and redness.

Based on the foregoing, there is a continuing need to formulate skin care compositions which improve the health and/or physical appearance of the skin, which are for example, aesthetically pleasing, stable, and effective in treating the appearance of wrinkles, fine lines, pores, poor skin color (e.g. redness, sallowness, and other forms of undesirable skin surface texture).

### SUMMARY OF THE INVENTION

Generally, the invention comprises hemp peptides, compositions comprising hemp peptides, including nutraceutical and personal care products, and methods of using such compositions as a treatment for skin and/or hair conditions, particularly human skin and/or conditions.

In one aspect, the invention comprises a composition comprising low-molecular weight, hemp-derived peptides and at least one excipient suitable for topical application to skin and/or hair. The peptides may be prepared by enzymatic hydrolysis of hemp protein. The excipients may be chosen to produce skin care products that include but are not limited to cleansers, toners, serums, creams, lotions and masks.

In another aspect, the invention comprises the use of a composition comprising low-molecular weight hemp-derived peptides to prevent, mitigate or treat skin conditions, including the prevention, mitigation or treatment of damage caused by free radicals and reactive oxygen species, ultraviolet light exposure, and/or environmental pollutants or irritants. In some embodiments, the composition may be used to promote healing of damaged, wounded or irritated skin. In some embodiments, the composition may be used to promote or accelerate the regeneration of skin, including, the renewal or regeneration of extracellular matrix or skin cells.

In another aspect, the invention comprises the use of a composition comprising hemp-derived peptides to repair, mitigate or improve animal hair conditions, including the repair, mitigation or improvement of damage caused by ultraviolet light exposure, and/or environmental pollutants or irritants, various hair treatments and styling products. In some embodiments, the composition may be used to promote healing and repair damaged hair. In some embodiments, the composition may be used to promote or accelerate the regeneration and repair of hair, including the renewal or regeneration of hair follicle, shaft and scalp.

In another aspect, the invention comprises the use of a composition comprising low-molecular weight, hemp-derived peptides to form a nutraceutical food or beverage that may improve the condition of skin and hair in subjects who consume the food or beverage. In some embodiments, the composition may be used to promote healing and repair of damaged skin and hair by delivering improved nutrition and key metabolites to the skin, scalp and hair cells to enhance or initiate healing and repair.

### BRIEF DESCRIPTION OF THE DRAWINGS

FIG. 1 is an SDS-PAGE gel showing protein bands from hemp protein hydrolysates.

FIG. 2 shows chromatograms obtained from UV absorbance detection at 220 nm and positive mode electrospray SCAN of a blank (0.1% TFA, 20 μL injection). Top is UV absorbance at 220 nm and bottom is mass spectrometric SCAN 250 to 2000 AMU.

FIG. 3 shows chromatograms obtained from UV absorbance detection at 220 nm and positive mode electrospray SCAN of sample 1002 (50 mg/mL in 0.1% TFA, 10 μL injection). Top is UV absorbance at 220 nm and bottom is mass spectrometric SCAN 250 to 2000 AMU.

FIG. 4 shows chromatograms obtained from UV absorbance detection at 220 nm and positive mode electrospray SCAN of sample 1003 (50 mg/mL in 0.1% TFA, 10 μL injection). Top is UV absorbance at 220 nm and bottom is mass spectrometric SCAN 250 to 2000 AMU.

FIG. 5 shows chromatograms obtained from UV absorbance detection at 220 nm and positive mode electrospray SCAN of sample 1004 (50 mg/mL in 0.1% TFA, 10 μL injection). Top is UV absorbance at 220 nm and bottom is mass spectrometric SCAN 250 to 2000 AMU.

FIG. 6 shows chromatograms obtained from ion extraction of signals associated with UV absorbance (top), mass spectrometric scan (second from top), phenylalanine (third from top), tyrosine (fourth from top) and tryptophan (bottom) from positive mode electrospray SCAN of sample 1002 (50 mg/mL in 0.1% TFA, 10 μL injection).

FIG. 7 shows mass spectrometric array obtained from sample 1002 at 26.3 minutes.

FIG. 8 shows mass spectrometric array obtained from sample 1002 at 31.4 minutes.

FIG. 9 shows mass spectrometric array obtained from sample 1002 at 21.0 minutes.

FIG. 10 shows mass spectrometric array obtained from sample 1002 at 25.2 minutes.

FIG. 11 shows a graph of results for fine lines (visual measurements), Code A vs. Code D.

FIG. 12 shows a graph of results for fine lines (visual measurements), Code B vs. Code D.

FIG. 13 shows a graph of results for coarse wrinkles (visual measurements), Code A vs. Code D.

FIG. 14 shows a graph of results for coarse wrinkles (visual measurements), Code B vs. Code D.

FIG. 15 shows a graph of results for cutometer measurements (R6 readings), Code A vs. Code D.

FIG. 16 shows a graph of results for cutometer measurements (R6 readings), Code B vs. Code D.

FIG. 17 shows a graph of results for cutometer measurements (R7 readings), Code A vs. Code D.

FIG. 18 shows a graph of results for cutometer measurements (R7 readings), Code A vs. Code D.

FIG. 19 shows a graph of results for TEWL measurements, Code A vs. Code D.

FIG. 20 shows a graph of results for TEWL measurements, Code B vs. Code D.

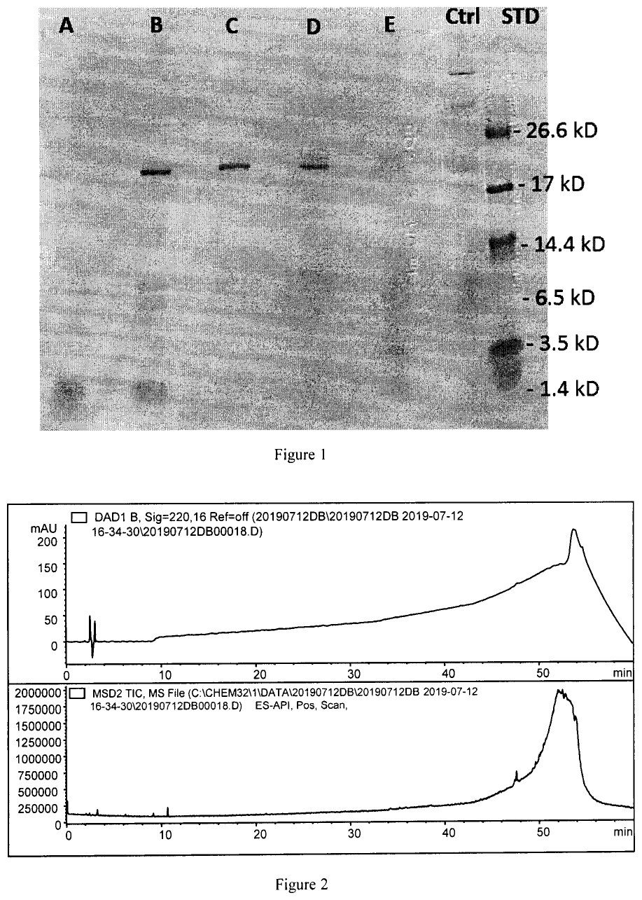

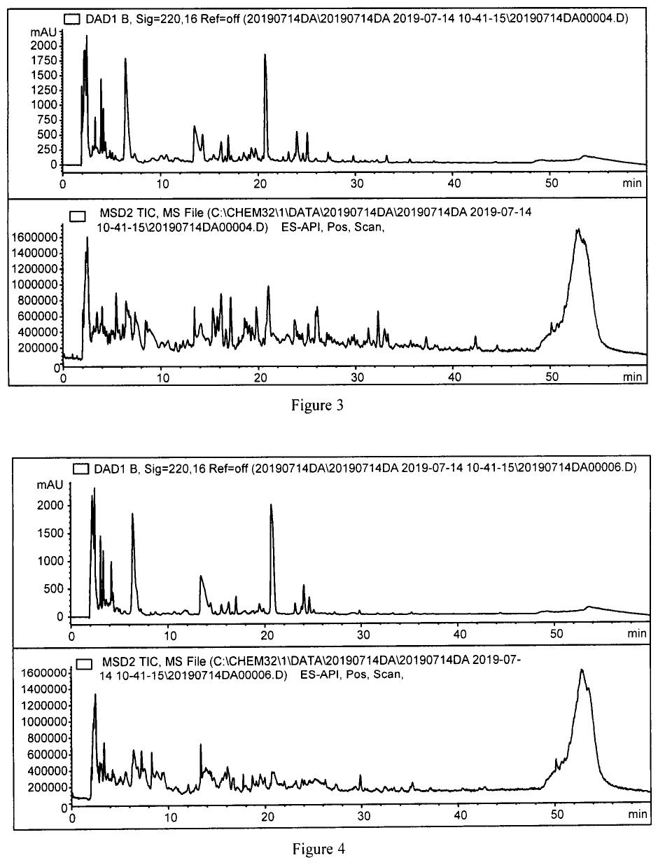

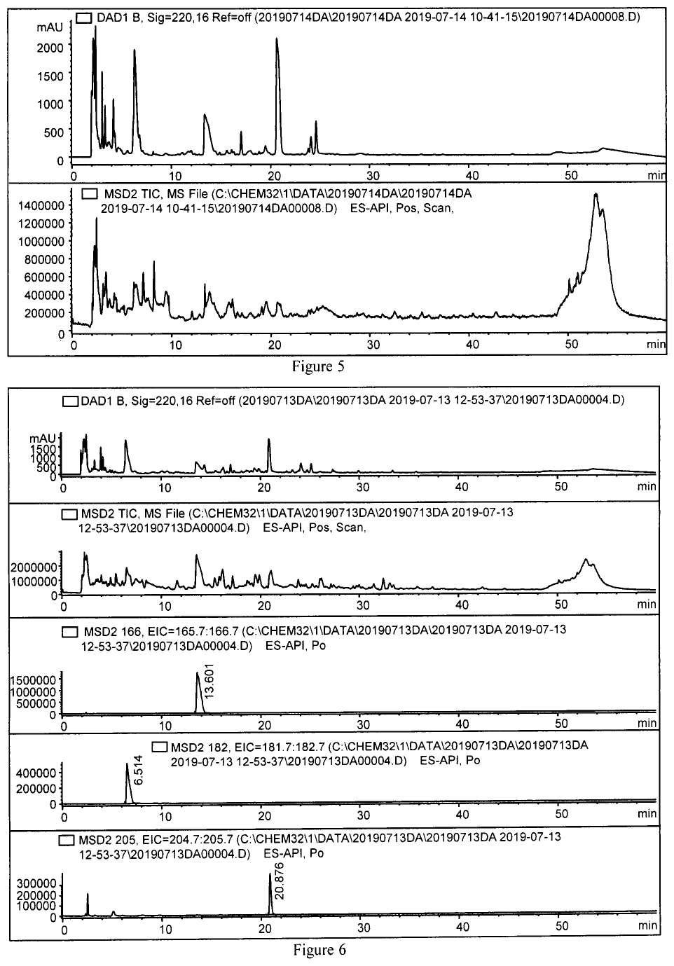

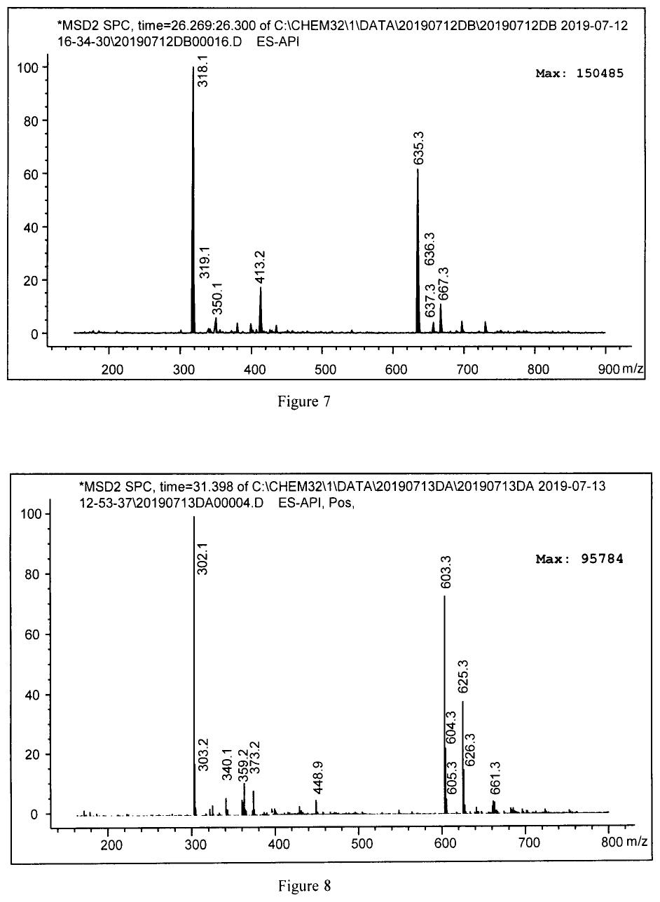

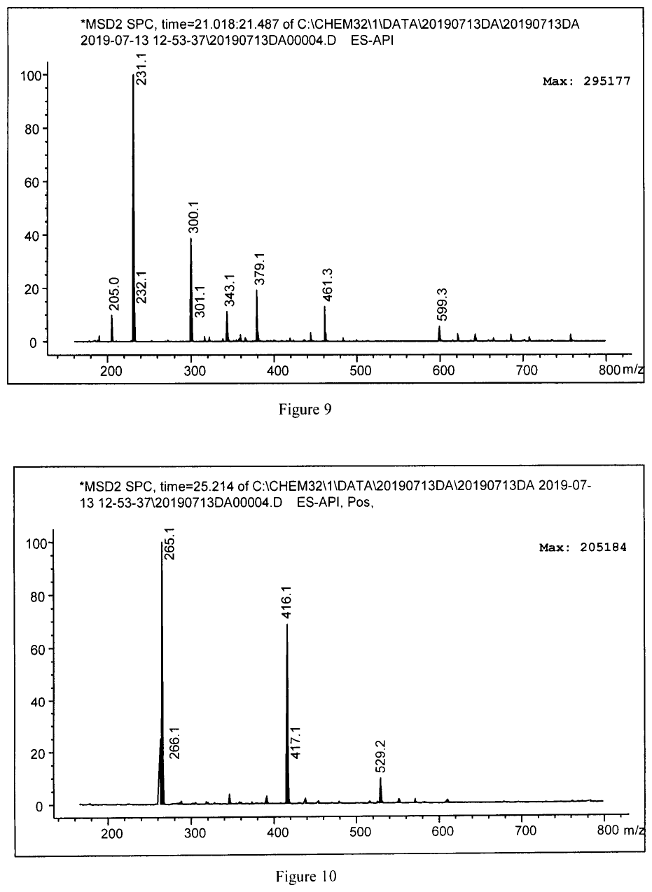

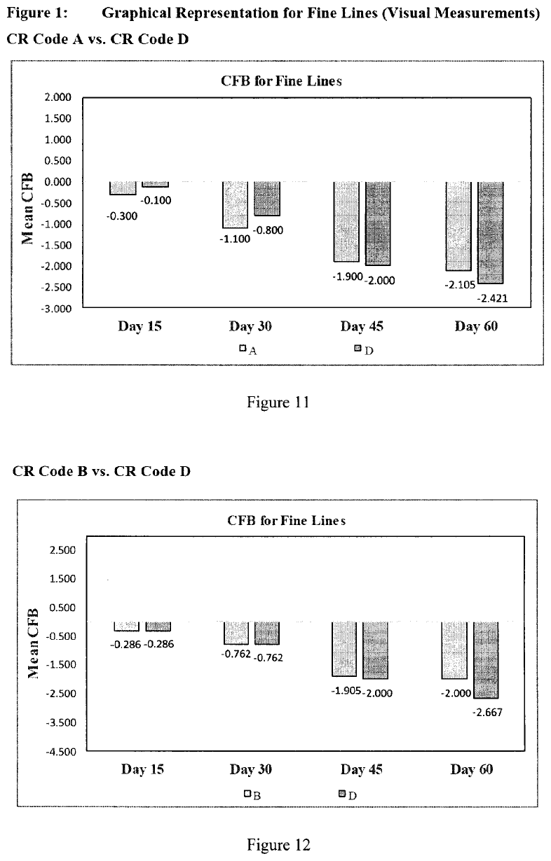

### DETAILED DESCRIPTION

Aspects of the present invention provide low-molecular weight peptides derived from hemp proteins, formulations or compositions comprising such peptides, and methods of using such peptides or formulations to improve skin and hair health, or treat skin and hair-related conditions or diseases, preferably with humans.

The term “LMWH peptides” means low-molecular weight hemp peptides derived from hemp proteins. As used herein, “low molecular weight” means having a molecular weight less than about 10 kDa, and preferably less than about 3 kDa, and most preferably less than about 1 kDA (1000 AMU). LMWH peptides may comprise or consist essentially of those peptides which pass through an ultrafiltration filter of a desired size. Molecular weight may be measured by any conventional technique, such as by SDS Polyacrylamide Gel Electrophoresis (SDS-PAGE) or chromatography such as HPLC-mass spectrometry, electrospray ionization (ESI) or matrix-assisted laser desorption ionization (MALDI) mass spectrometry. In some embodiments, the LMHW peptides comprise 2 to 7 amino acids.

In some embodiments, a composition of the present invention comprises a peptide mixture wherein at least 10%, 20%, 30%, 40%, 50%, 60%, 70%, 80%, or 90% of peptides (by mass) are LMWH peptides.

The term “skin” includes the keratin-containing layers disposed as the outermost protective covering of mammals (e.g., humans, dogs, cats, etc.). The term “hair” includes the protein filament that grows from follicles found in the dermis of the skin. The term “topical application”, as used herein, means to apply or spread the compositions of the present invention onto the surface of skin and hair.

Industrial hemp (Cannabis sativa L.) provides commercially valuable hemp fibre, which is a lignocellulosic fibre with many uses. Hemp seed is currently a byproduct of fibre processing, and is a rich source of high quality oil and protein. Hemp protein hydrolysates have been investigated for anti-oxidant properties, amongst other potentially beneficial effects. Cannabis is of course the source of cannabinoids, which are compounds which act on cannabinoid receptors in cells, and which can alter neurotransmitter release in the brain. The present invention is based, not on cannabinoids, but on peptides produced by hydrolyzing proteins extracted from a cannabis plant. As used herein, a cannabis plant includes a Cannabis sativa plant, some varieties of which are known as industrial hemp, as well as other species or varieties which produce psychoactive and non-psychoactive cannabinoids, including Cannabis indica and Cannabis ruderalis.

The present invention relates to the surprising discovery that LMWH peptides, when applied to the skin and hair, or consumed as a nutraceutical product, may result in improvement of skin and hair health. This improvement may be evidenced by the visible reduction or elimination of fine lines or wrinkles, or any other visible skin aging and environmental damage, or by a halt or reversal in the reduction in the cells and blood vessels that supply the skin, or flattening of the dermal-epidermal junction which results in weaker mechanical resistance of this junction. The improvement in skin and hair health also manifests itself as an amelioration or reversal of symptoms of skin and hair diseases or conditions.

Bioactive peptides may be physiologically active components. These biologically active peptides may be utilized to yield health benefits. Bioactive peptides may be inactive within the parent protein sequence. However, bioactive peptides are released from the parent protein sequence upon hydrolysis and once released, they may exert physiological effect on the human body.

In some embodiments, LMWH peptides may be derived from hemp proteins extracted or purified from hemp seeds in a conventional manner. Hemp protein comprises primarily two globular proteins: 60-80% edestin having a molecular weight about 50 kDa, and the remainder albumin having a molecular weight of about 65-70 kDa. The hemp proteins are hydrolyzed, by acid, alkaline, thermal or enzymatic hydrolysis, or combinations thereof, to produce LMWH peptides.

The hydrolysate may be produced by the use of various proteases, such as endopeptidases such as a serine protease (for example Alcalase™), exopeptidases, or combinations thereof (for example Flavourzyme™). Many proteases are commercially available with well-known properties and mechanisms of action.

In other embodiments, the hemp protein may be hydrolyzed with chemical (acid or alkaline) hydrolysis, or thermal hydrolysis, or chemical thermal hydrolysis.

The hydrolysate may be passed through filters, such as ultrafiltration filters, to purify or isolate a desired low molecular weight fraction. For example, the hydrolysate may be passed through a series of filters to achieve a fraction which comprises, or consists essentially of LMHW peptides. Suitable filters may include membrane filters or gel filters, or any other known filter which physically separates peptides based on molecular size.

In other embodiments, the hydrolysate may be fractionated with chromatographic methods, such as liquid chromatography or another similar method, or electrophoretic methods, such as SDS-PAGE, all of which is well known to those skilled in the art.

In some specific embodiments, the LMWH peptides comprise 2 to 6 amino acids, and may comprise without limitation, at least one, any combination of some, or all of the peptides shown in the Table below. Any other LMWH peptide sequence derived from hemp protein may also be utilized.

| LC/MS Retention Time | | |
|---|---|---|
| of Peptide Elution | Peptide Amino Acid Sequence | SEQ ID NO. |
| 6.316 | PETF | 1 |
| 11.593 | ASPR | 2 |
| 13.346 | WS | |
| 15.178 | PSR | |
| 15.648 | RPS | |
| 15.961 | MAR | |
| 16.947 | ARSQ | 3 |
| 19.343 | NDNA | 4 |
| 19.812 | SRDN | 5 |
| 19.812 | IQNT | 6 |
| 19.812 | NQA | |
| 20.939 | YNL | |
| 20.939 | LKY | |
| 21.018 | YNL | |
| 21.018 | LIAF | 7 |
| 23.617 | RARS | 8 |
| 25.214 | LRQN | 9 |
| 25.809 | VTRSGA | 10 |
| 27.124 | HAC | |
| 29.973 | LQNRDI | 11 |
| 31.398 | PGCPET | 12 |
| 32.212 | VSEYW | 13 |
| 32.685 | VSEYW | 14 |
| 32.465 | ERGTSS | 15 |

LMWH peptides, preferably isolated or purified, may be formulated in a composition to be applied topically to skin or to be ingested. The composition may comprise any other ingredient which may be useful in skin and hair care, such as other active ingredients or non-medicinal ingredients.

In some embodiments, the peptide concentration may be between 0.05 and 20.0% by weight, preferably between 0.1 and 5.0%, and most preferably between 0.5 and 2.0%.

Certain compositions of the present invention may contain LMWH peptides and a dermatologically acceptable carrier, which can be in a wide variety of forms. For example, emulsion carriers, including, but not limited to, oil-in-water, water-in-oil, water-in-oil-in-water, and oil-in-water-in-silicone emulsions, are useful. The carrier can also consist of a combination of surfactants that provide the desired characteristics. The term “dermatologically-acceptable,” as used herein, means that the compositions or components thereof so described are suitable for use in contact with mammalian skin and hair without undue toxicity, incompatibility, instability, allergic response, and the like. The term “safe and effective amount” as used herein means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of one skilled in the art.

Preferred carriers contain an emulsion such as oil-in-water emulsions, water-in-oil emulsions, and water-in-silicone emulsions. A given component will distribute primarily into either the water or oil/silicone phase, depending on the water solubility/dispersibility of the component in the composition. Oil-in-water emulsions are especially preferred.

Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.01% to about 10%, more preferably from about 0.1% to about 5%, of an emulsifier, based on the weight of the carrier. Emulsifiers may be nonionic, anionic or cationic. The emulsion may also contain an anti-foaming agent to minimize foaming upon application to the keratinous tissue. Anti-foaming agents include high molecular weight silicones and other materials well known in the art for such use.

The compositions herein may include another skin or hair care active ingredient, as well as a wide variety of other optional ingredients, such as one or more of a conditioning agent, a structuring agent, a thickening agent, or a surfactant.

Conditioning agents may be selected from humectants, moisturizers, or conditioners, which may be biologically active. Preferably, the conditioning agent is selected from urea, guanidine, sucrose polyester, panthenol, dexpanthenol, allantoin, or bioactives, and combinations thereof.

Structuring agents are particularly preferred in embodiments comprising an oil-in-water emulsion. Without being limited by theory, it is believed that the structuring agent assists in providing rheological characteristics to the composition which contribute to the stability of the composition. For example, the structuring agent tends to assist in the formation of the liquid crystalline gel network structures. The structuring agent may also function as an emulsifier or surfactant. Preferred structuring agents are those having an HLB of from about 1 to about 8 and having a melting point of at least about 45° C.

Preferred structuring agents of the present invention may be selected from stearic acid, palmitic acid, stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. More preferred structuring agents of the present invention are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. Even more preferred structuring agents are selected from stearic acid, palmitic acid, stearyl alcohol, cetyl alcohol, behenyl alcohol, steareth-2, and mixtures thereof.

In some embodiments, the composition may comprise one or more thickening agents, such as carboxylic acid polymers, crosslinked polyacrylate polymers, polyacrylamide polymers, polysaccharides, gums.

In some embodiments, the composition may also comprise of one or more surfactants such as cocoamidyl betaine, sodium lauryl and laureth sulphate, sodium lauryl sulphoacetate, decyl glucoside and the like.

Embodiments of the present invention may be useful for regulating visible and/or tactile discontinuities in mammalian skin, including discontinuities in skin texture and color. For example, as the apparent diameter of pores decreases, the apparent height of tissue immediately proximate to pore openings approaches that of the interadnexal skin, the skin tone/color becomes more uniform, and/or the length, depth, and/or other dimension of lines and/or wrinkles are decreased.

Embodiments of the present invention may also be useful for regulating the condition of skin. Regulation of skin condition, in particular human skin condition, is often required due to conditions which may be induced or caused by factors internal and/or external to the body. Examples include, environmental damage, radiation exposure (including ultraviolet radiation), chronological aging, menopausal status (e.g., post-menopausal changes in skin), stress, diseases, etc. For instance, “regulating skin condition” includes prophylactically regulating and/or therapeutically regulating skin condition, and may involve one or more of the following benefits: thickening of skin (i.e., building the epidermis and/or dermis and/or sub-dermal (e.g., subcutaneous fat or muscle) layers of the skin and where applicable the keratinous layers of the nail and hair shaft) to reduce skin atrophy, increasing the convolution of the dermal-epidermal border (also known as the rete ridges), preventing loss of skin elasticity (loss, damage and/or inactivation of functional skin elastin) such as elastosis, sagging, loss of skin recoil from deformation; non-melanin skin discoloration such as under eye circles, blotching (e.g., uneven red coloration due to, e.g., rosacea) (hereinafter referred to as “red blotchiness”), sallowness (pale color), discoloration caused by telangiectasia or spider vessels.

The term “sagging” as used herein means the laxity, slackness, or the like condition of skin that occurs as a result of loss of, damage to, alterations to, and/or abnormalities in dermal elastin. The terms “smoothing” and “softening” as used herein mean altering the surface of skin such that its tactile feel is improved. “Signs of skin aging” include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles and coarse deep wrinkles, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

Compositions of the present invention are also of relevance to applications on hair and scalp. Such compositions for application on scalp or hair generally provide benefits of providing strength to the hair fibre, improvement to the visual appearance, texture and thickness of the hair. Regulation of hair conditions, namely mammalian and in particular human hair condition, is often required due to conditions which may be induced or caused by factors internal and/or external to the body. Examples include, chemical treatments with bleaching solutions and dyes, hair curling and straightening products, heat treatments, environmental damage, radiation exposure (including ultraviolet radiation), chronological aging, stress, diseases, etc. Hair care compositions are delivered in the form of hair oils, hair care gels, tonics, masks and creams and also in the form of wash off products like shampoos and conditioners.

Prophylactically regulating skin and hair conditions includes delaying, minimizing and/or preventing visible and/or tactile discontinuities in the skin and hair (e.g., texture irregularities of the skin and hair which may be detected visually or by feel). As used herein, therapeutically regulating skin condition includes ameliorating, e.g., diminishing, minimizing and/or effacing, discontinuities in skin.

The compositions of the present invention may also be useful for improving skin and hair appearance and/or feel. For example, compositions of the present invention are useful for regulating the appearance of skin and hair condition by providing an immediate visual improvement in appearance following application of the composition to the skin and hair. Generally speaking, compositions of the present invention which further contain particulate materials will be most useful for providing the immediate visual improvement.

The compositions of the present invention may provide additional benefits, including stability, absence of significant (consumer-unacceptable) skin and hair irritation and good aesthetics.

The compositions of the present invention are stable. The ingredients used herein, including the LMWH peptides are stable in the composition and are compatible with each other and with the other skin and hair care actives, including without limitation, niacinamide, phytantriol, farnesol, bisabolol, and salicylic acid. Therefore, the compositions containing the combination of LMWH peptides are capable of providing additive and/or synergistic skin and hair benefits. Additionally, the resulting personal care compositions have commercially acceptable product stability and shelf-life.

The compositions containing LMWH peptides preferably have good aesthetics. Examples of good aesthetics include compositions, such as creams, lotions shampoos and conditioners that (i) are light and nongreasy, (ii) have a smooth, silky feel upon the skin, (iii) spread easily, and/or (iv) absorb quickly. The compositions preferably are preferably also able to clean and condition the hair and scalp. Other examples of good aesthetics include compositions that have a consumer acceptable appearance (i.e. no unpleasant odor or discoloration present), and provide good skin feel.

As will be apparent to those skilled in the art, these and other elements of the composition may be combined to produce a suitable paste, lotion, cream, gel, ointment, or balm suitable for topical application.

In another aspect, the composition of this invention provides a nutraceutical, food product or beverage for improving skin and hair quality, particularly by improving hydration, elasticity and tension of both epidermis and dermis of the skin through the synergistic effects exerted by its ingredients and by means of the administration route. In addition to the LMWH peptides, the beverage composition can comprise one or more ingredients selected from but not limited to, collagen (such as fish collagen peptides), hydrolyzed collagen, hyaluronic acid, flavouring agents, colourants, sweeteners, fruit juice concentrates and acidulants. The beverage composition can also comprise other ingredients such as vitamins, minerals, additives, antioxidants, carbohydrate sources, amino acids and trace elements. In some embodiments, the beverage may be an alcoholic beverage.

The beverage may comprise LMWH peptides between about 0.1% to about 5% by weight, and preferably about 0.2% to about 2.0%.

The beverage composition of the present invention is primarily applicable in a non-medical cosmetic method. Suitably, the present invention further provides use of a beverage composition for improving skin conditioning, skin hydration, skin nourishment and skin appearance. It can be used to reduce the signs of ageing and could be used by individuals who so desire.

### EXAMPLES

The following examples are intended to illustrate specific embodiments of the invention described herein, and not be limiting of the claimed invention in any way.

### Example 1: Production of LMWH Peptides

A 5% (w/w) solution of hemp protein was prepared by mixing Organic HempSol-O80™ (Lot #OS080HPP060318na) hemp protein with water. To individual samples of the hemp protein solution was added the following three commercial protease enzymes: 1) a cocktail of Alcalase enzyme from Bacillus lichemiformis and Flavourzyme from Aspergillus oryzae, 2) M Amano SD from Aspergillus oryzae and 3) A Amano 2SD.

The protease enzymes were added at a concentration of 8% (wt./protein powder wt.) to the hemp protein solution. The hemp protein hydrolysis mixtures were incubated at the optimal temperature of 50° C. The optimal pH for each of the enzymes was maintained by adding either sodium hydroxide or hydrochloric acid to the incubation mixtures. The mixtures were incubated for 8 hours. When using the enzyme cocktail, the Alcalase protease was incubated with the hemp protein for the first 6 hours, and the second Flavourzyme protease was then added and incubated for the last 2 hrs.

TABLE 1
The information of enzymes used in this study

| | | | | Optimum | Optimum |
|---|---|---|---|---|---|
| Sample ID | Enzyme | | Supplier | Temperature | pH |
| NL20180710 A | Enzyme | Protease from Bacillus | Sigma-Aldrich | 50° C. | 8.30 |
| | Cocktail | lichemiformis (Alcalase) | | | |
| | | Protease from Aspergillus | Sigma-Aldrich | 50° C. | 5.0-5.5 |
| | | oryzae (Flavourzyme) | | | |

| | | | | |
|---|---|---|---|---|
| NL20180710 B | Protease A “Amano” 2SD, from | Amano Enzyme Inc. | 50° C. | 7.0 |
| | Aspergillus oryzae | | | |
| NL20180710 C | Protease M “Amano” SD, from | Amano Enzyme Inc. | 50° C. | 6.0 |
| | Aspergillus oryzae | | | |
| NL20180710 D | ProteAX, from Aspergillus oryzae | Amano Enzyme Inc. | 70° C. | 7.0 |
| NL20180710 E | Thermoase GL-30 | Amano Enzyme Inc. | 70° C. | 7.5 |

TABLE 2
The information of enzymes used in this study

| Sample ID | NL20180710 A | | NL20180710 B | NL20180710 C | NL20180710 D | NL20180710 E |
|---|---|---|---|---|---|---|
| Enzyme | Alcalase + | | Protease A | Protease M | ProteAX | Thermoase GL-30 |
| | Flavourzyme | | “Amano” 2SD | “Amano” SD | | |
| pH | 8.27 | 5.54 | 6.8 | 5.97 | 7.05 | 7.65 |
| Temp | 52.1 | 52.2 | 50.8 | 49.9 | 69.2 | 68.9 |

| | | | | | |
|---|---|---|---|---|---|
| HempSol (g) | 5.01 | 5 | 5.01 | 5.01 | 5.02 |
| Water (g) | 95.22 | 95.14 | 95.15 | 95.1 | 95.07 |
| Protein (g) | 3.63 | 3.63 | 3.63 | 3.63 | 3.64 |
| Solid (g) | 4.79 | 4.78 | 4.79 | 4.79 | 4.80 |

Centrifuge at 5000 × g for 6 min

| Centrates | NL20180710 A-C | NL20180710 B-C | NL20180710 C-C | NL20180710 D-C | NL20180710 E-C |
|---|---|---|---|---|---|
| Total (g) | 97.49 | 96.36 | 94.53 | 96.09 | 93.38 |
| Solid (g) | 5.38 | 4.69 | 4.12 | 4.84 | 4.06 |
| Protein (g) | 3.23 | 3.26 | 2.98 | 3.38 | 3.08 |
| Protein | 88.81% | 89.83% | 81.99% | 92.93% | 84.56% |
| Retained % | | | | | |

After 8 hours of incubation, the enzyme incubation mixtures were centrifuged at 5,000 g to sediment any insoluble material. The resulting solutions were successively filtered through four ultrafiltration membranes with molecular cutoffs of 50, 30, 10 and 3 kDaltons, using commercially available ultrafiltration centrifuge cartridges, according to the manufacturers protocols. The material that passed through the final 3 kDa membrane yielded peptide test sample solutions which were then freeze dried to yield the test peptide samples. The enzyme cocktail of Alcalase enzyme from Bacillus lichemiformis and Flavourzyme from Aspergillus produced peptide mixture 1002, the M Amano SD enzyme produced peptide mixture 1003 and A Amano 2SD yielded peptide mixture 1004.

### Peptide Samples Peptide Report

Peptide samples 1002, 1003 and 1004 were each dissolved completely in 0.1% trifluoroacetic acid (TFA), (50 mg/mL) and 10 μL of these solutions were injected onto the LC/MS apparatus. Several gradient elution separations were tried using mixtures of 0.1% TFA in water and 0.1% TFA in acetonitrile. After establishing a reasonable elution profile using the samples, several mass spectrometric mass ranges were utilized to obtain signals.

### Chromatographic Equipment

A HP 1100 HPLC system equipped with a refrigerated autosampler, column heater and DAD and LC/MS detectors was used. The initial chromatographic conditions were as follows: Chromatographic conditions for hemp protein digest (positive mode), Column used was the Phenomenex Luna 3μ C18(2) 100 A 15 cm×4.60 mm equipped with a guard column (SecurityGuard C18). Column heater set at 40° C. Mobile phase A: 0.1% trifluoroacetic acid in 18 mega Ω water. Mobile phase B: 0.1% trifluoroacetic acid in HPLC grade acetonitrile. Flow characteristics were 0.8 mL/min. Gradient used included at time=0 min, 4% mobile phase B; time=5 min, 4% mobile phase B; time=45 min, 30% mobile phase B; time=50 min, 95% mobile phase B; time=58 min, 4% mobile phase B; time=60 min, 4% mobile phase B. There was a 5 min post time interval. The DAD detector conditions included: specific signals are collected at 214 nm, 220 nm, 250 nm and 280 nm. All spectra were stored from 190 nm to 400 nm with a range step of 2 nm electrospray mass spectrometer conditions included: positive mode, gas temp 350° C., drying gas 12 L/min, neb pressure 35 psig, vaporizer 350° C. and capillary voltage 3000 V. The scan conditions included: low mass 160, high mass 800, fragmentor 70, gain 1, threshold 150, stepsize 0.10 (method 3) and low mass 250, high mass 2000, fragmentor 70, gain 1, threshold 150, stepsize 0.10 (method 4).

### Chromatographic Results

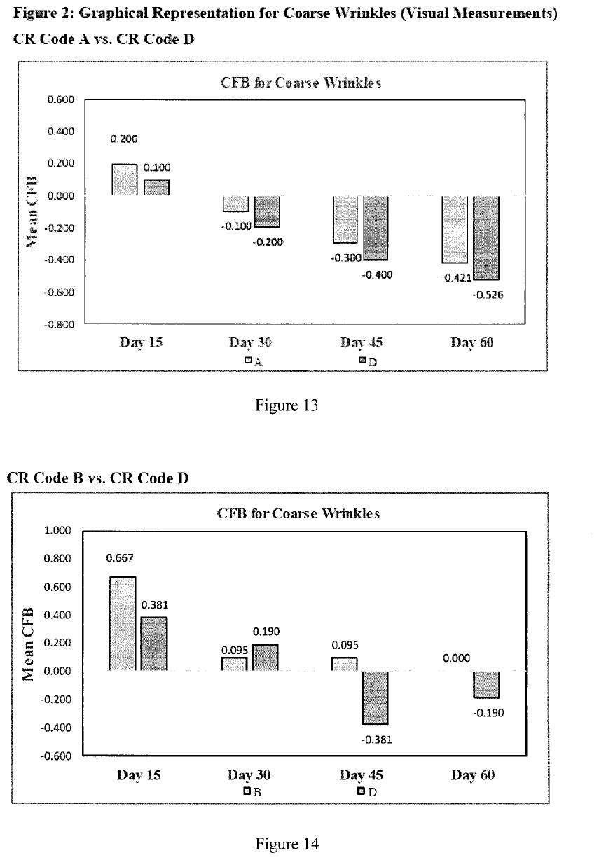

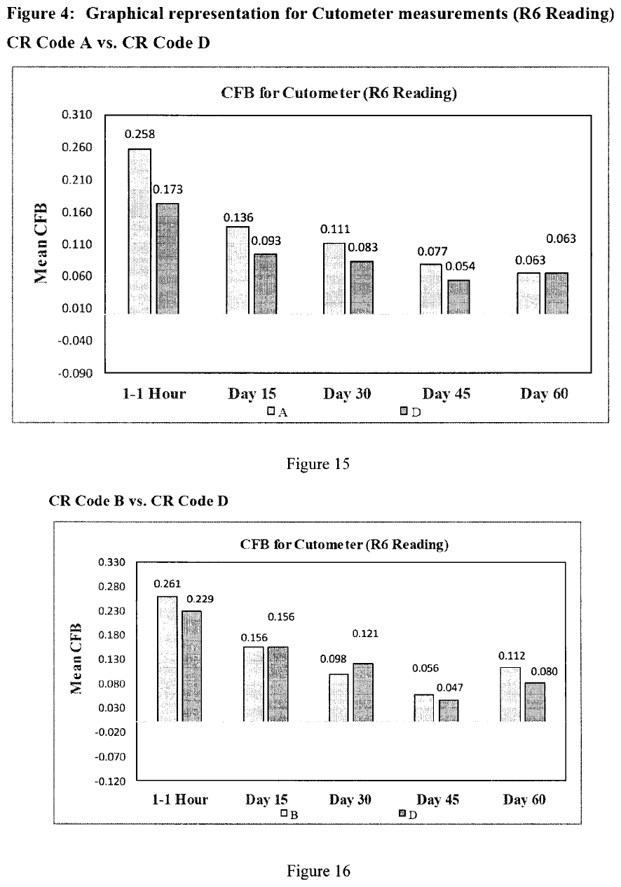

Representative chromatograms obtained from UV absorbance at 220 nm and a positive mode electrospray mass spectrum for blanks and each sample are presented in FIGS. 2, 3, and 4. No intact large protein was seen in any of the samples.

All samples provided relatively similar HPLC profiles in UV absorbance and mass spectrometric detection. Sample 1002 was slightly different in profile in that it had several more obvious peaks eluting in the 15 minute to 34 minute range on the mass spectrometric profile. Sample 1002 was used to examine selected mass spectrometric peaks and to extract ions of interest.

### UV Absorbance and Mass Spectral Interpretation of Samples

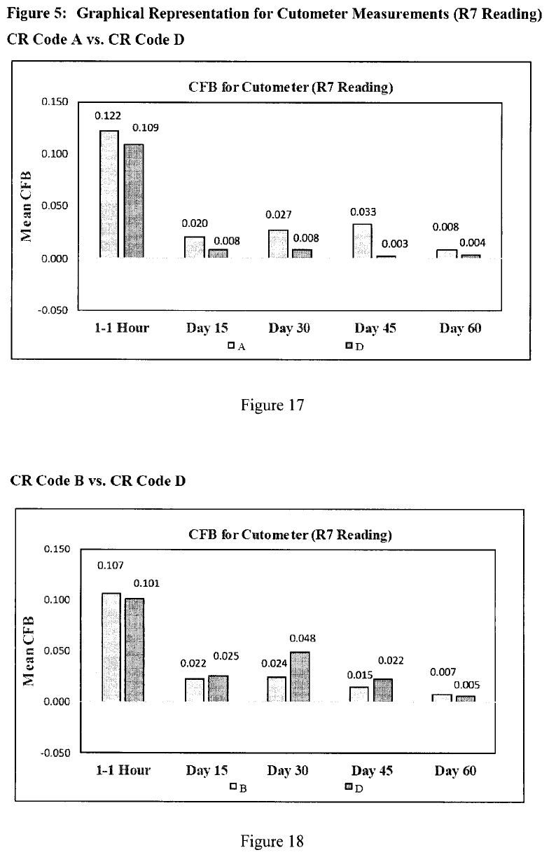

FIG. 5 shows chromatograms obtained from UV absorbance detection at 220 nm and positive mode electrospray SCAN of sample 1004 (50 mg/mL in 0.1% TFA, 10 μL injection). Top is UV absorbance at 220 nm and bottom is mass spectrometric SCAN 250 to 2000 AMU.

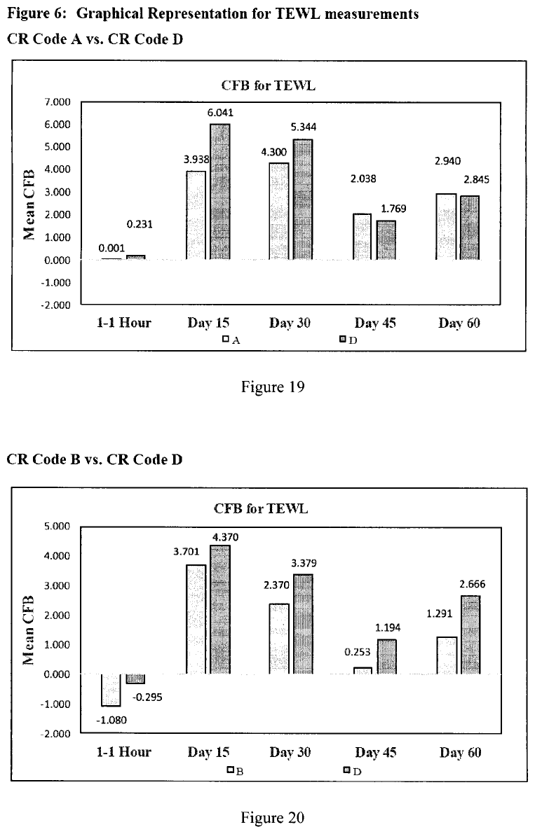

Both the UV absorbance and SCAN mass spectral traces indicated a large number of separated compounds. Of note were several with trailing profiles that were identified to be individual amino acids such as phenylalanine, tyrosine and tryptophan. These aromatic amino acids provide UV absorbance and mass spectrometric signals and in the low salt mobile phases required for electrospray mass spectroscopy provided tailing peaks. Ion extraction of the masses for these three amino acids found them to be present and they provided significant signals (FIG. 6). This suggests that the sample is highly digested and has released a substantial amount of free amino acids.

The presence of large amounts of amino acids suggests that the original proteins/peptides may have been substantially digested. Further analysis of the mass spectra suggested that no intact proteins and few intact larger peptides remained. Signals for very small peptides were determined (both MH⁺ or MNa⁺ as well as 2MH⁺ or 2MNa⁺ and M2H⁺ or MHNa⁺) suggesting that the samples contain a significant amount of Na⁺ salts.

Intact proteins provide an array of signals consisting of multiple charges. No such signal array was observed with the peptide samples.

The peptide samples provide numerous mass spectrometric signals indicating peptide fragments of much less than 800 AMU and a few between 800 and 1200 AMU indicating very complete digestion of the samples. Prominent signals seen in the peptide samples were at MW 489 and 603 were similar to those observed by Das et al.

With respect to FIG. 9, the peaks could be interpreted as MH⁺ 759, MNa⁺ 781 and M2H⁺ 380 with molecular weight roughly around 758. With respect to FIG. 8, the peaks could be interpreted as MH⁺ 603, MNa⁺ 625 and M2H⁺ 302, with molecular weight roughly around 602 or 301.

With respect to FIG. 9, this could be a mixture of two peptides Peptide 1 could be MH⁺ 599, MNa⁺ 622 and M2H⁺ 300, with molecular weight roughly around 598. Peptide 2 could be MH⁺ 461, MNa⁺ 483 and M2H⁺ 231, with a molecular weight roughly around 460.

With respect to FIG. 10, the peaks could be interpreted as MH⁺ 529, and M2H⁺ 265, with molecular weight roughly around 528. The mass at 416 might have been a fragment or a co-eluting compound.

No large proteins or peptides were determined in the peptide samples. The peptide samples were qualitatively similar but sample 1002 appeared to have more peptide-like signals in it than the other two samples. Sample 1002 provided mass spectral signals for a number of individual amino acids as well as many signals for what appear to be peptides ranging from dipeptides to peptides of molecular weights up to 1000 AMU.

Example 2: This study was conducted to understand how gene expression in the skin is influenced by specific test materials, which were peptide samples 1002 and 1004. The study was performed using a full-thickness in vitro skin culture model containing epidermal and dermal cell layers (EFT-400, MatTek). Gene expression was assessed in the full-thickness tissues following a 24 hour exposure to the TMs and the following treatment groups were included in the study (N=4):
Hemp protein hydrolysate sample 1002, 2% in DPBS
Hemp protein hydrolysate peptide sample 1004, 2% in DPBS
DPBS [Vehicle Control]

Table 3 highlights results where the change in gene expression was at least 1.5-fold up or down; results are sorted & shaded by gene function, then alphabetically by gene.

TABLE 3
Statistically Significant Changes in Expression versus the Vehicle Control*

| Gene | 2% NL20181002 | 2% NL20181004 | Gene Function |
|---|---|---|---|
| EDN1 | 1.97 | n.s. | growth factor/pigmentation |
| HBEGF | n.s. | 1.55 | growth factor/wound healing |
| ICAM1 | 1.78 | n.s. | growth factor/wound healing |
| MT2A | 1.74 | n.s. | antioxidant/stress response |
| SOD2 | 1.69 | n.s. | antioxidant/stress response |
| CSF2 | 3.09 | n.s. | inflammation/immune response |
| CXCL8/IL8 | 2.73 | n.s. | inflammation/immune response |
| IL10 | 2.23 | n.s. | inflammation/immune response |
| IL1A | 1.60 | n.s. | inflammation/immune response |
| IL1B | 2.32 | n.s. | inflammation/immune response |
| IL6 | 1.87 | n.s. | inflammation/immune response |
| TNFa | 3.82 | 2.46 | inflammation/immune response |
| FLG | n.s. | −1.56 | epidermal barrier maintenance |
| LCE3D | 2.00 | 1.77 | epidermal barrier maintenance |
| MMP1 | 2.20 | n.s. | extracellular matrix breakdown |

*linear fold-change values ≥2.0-fold are listed in bold; n.s. = not statistically significant

The 2% 1002 sample regulated more genes than the 2% peptide sample 1004 at the 24 hour time period. The 2% 1002 produced changes in gene expression that correspond with beneficial effects in the skin namely increased expression of genes related to antioxidant activity (MT2A, SOD2) and increased expression of genes related to extracellular matrix turnover (MMP1).

Both samples 1002 and 1004 increased expression of inflammatory genes (CSF2, IL1A, IL1B, IL6, IL8, IL10, TNFa). Transient inflammation is not necessarily negative, as it is necessary to initiate wound healing, cell renewal and turnover, etc. This is consistent with the increase in genes associated with wound healing (HBEGF and ICAM1).

The 2% 1002 TM increased expression of a growth factor that regulates skin pigmentation (EDN1) at 24 hours.

Gene expression results are impacted by the time points selected in the study design, for example, the length of exposure to the samples. Depending on the gene, changes in expression can occur as early as two hours and as late as several days. Twenty-four hours is a standard exposure time for many in vitro studies. Analysis at additional time points may reveal additional changes in gene expression, or show regulation in a different direction.

In this study, gene expression was analyzed in the full-thickness tissue. Additional analysis of the separate layers, for example, epidermis and/or dermis, may identify additional changes in gene expression, or enhance the fold-change values of those already regulated, that may have been masked by analyzing both cell types together.

Example 3: Cytotoxicity was assessed following the 24 hour exposure to each test material. Control cultures were included to provide a Low LDH (−) and a High LDH (+) signal: one culture was treated with 1% Triton X-100™ to serve as the (+) LDH control, and two untreated cultures served as the (−) LDH controls. Cytotoxicity was less than 5% for each TM group, indicating healthy tissues for gene expression analysis.

The test materials for the study were provided as a powder. Each material was stored at room temperature, shielded from light, until use. The day of treatment, an aliquot of each peptide powder was prepared as a 2% solution (20 mg/mL) in a DPBS vehicle. Following a brief vortexing step, each peptide powder went into solution readily with the DPBS vehicle. Each TM was sterile-filtered prior to application. The tissue was equilibrated using EFT-400 tissues (Mattek™) overnight at 37° C. with 5% CO₂ and ˜95% relative humidity. The following day, equilibration medium was removed from each well and replaced with 2.5 mL fresh maintenance medium. The treatment and maintenance of cultures included each tissue receiving a 25 uL topical application of one TM, with 4 biological replicates per group. A sterile glass spreader was used to distribute the TM across the surface. For the LDH (+) tissue [N=1], a 100 uL volume of 1% Triton X-100 was applied to the surface of one EFT culture, and 2 EFT cultures were left Untreated as the LDH (−) tissues. Each culture was visually inspected to ensure the even distribution of topical treatment.

Following the application of TMs, cultures were returned to the incubator at 37° C. with 5% CO₂ and ˜95% relative humidity for 24 hours. After 24 hours, test medium from each well was collected for an LDH assay (described below). The TM was then washed from the surface of each culture with sterile Dulbecco's phosphate buffered saline (DPBS). Following the removal of the TM, each culture was cut into quarters and placed into a tube containing RNAlater™ preservative solution for a 1-2 hour incubation at room temperature, then moved into 4° C. Following a four day incubation in RNAlater™ at 4° C., RNA was isolated from each tissue.

The LDH cytotoxicity assay included each sample of collected culture medium being diluted 1:10 with sterile PBS. A background control (diluted EFT medium that was not used for cell culture), a “Low Control” (diluted EFT medium collected from the Untreated EFT culture wells), and a “High Control” (diluted EFT medium collected from 1% Triton X-100 treated culture well) were included in the assay; each sample was added to an optically clear, flat-bottom 96-well plate, with duplicate technical replicates.

The LDH reaction mixture (Takara™) was prepared and added to each aliquot of diluted medium (1:1). The reaction plate was then incubated for roughly 25 minutes at room temperature and was protected from light. Stopping solution (1.0N HCl) was then added to each well and absorbance was measured at 492 nm with a reference filter at 620 nm. Each sample absorbance value was calculated as the mean OD492-OD620 value for the duplicate reaction wells, with the blank absorbance value subtracted. The % cytotoxicity was then calculated relative to the untreated (negative control, set to 0% cytotoxicity) and the Triton X-100 treated (positive control and set to 100% cytotoxicity) absorbance values, according to kit instructions: % Cytotoxicity=[(Test Media Value−Low Control)/(High Control−Low Control)]*100. The measured % cytotoxicity was less than 5% for each group.

The RNA isolation included RNA being isolated from ¼ of each tissue using a Maxwell 16 Simply RNA Tissue kit, Promega™, following the manufacturer's instructions. RNA samples under 200 ng/uL at isolation, the concentration recommended for OpenArray™ processing, were vacuum concentrated to be greater than 200 ng/uL. Vacuum concentration was not necessary for samples with RNA greater than 200 ng/uL. RNA concentration and purity were determined using a Nanodrop 2000 spectrophotometer. All samples shown in Table 4 with high-quality RNA metrics and with similar yields for each group. The cDNA was generated using a High Capacity cDNA Synthesis Kit according to the manufacturer's instructions (Applied Biosystems™). For OpenArray processing of 112 Standard Skin Panel™ genes, cDNA was generated from 2000 ng RNA per sample. The qPCR reactions were run using validated Taqman™ gene expression assays. OpenArrays were run in a Life Technologies QuantStudio 12K Flex™ instrument. Each gene was assayed in duplicate. The qPCR data quality was assessed and exported from the raw data files using Expression Suite™ software (Life Technologies™). The qPCR data was then imported into the OmicsOffice for qPCR™ tool of TIBCO Spotfire Analyst™ software. Statistical analysis was performed using the relative quantitation (RQ) method. In the first step of an RQ analysis, the CT value of the target gene was normalized to the CT value of an endogenous control gene to generate the delta CT (dCT). The dCT values were calculated in order to normalize for variability between the samples that may occur during the experimental procedures.

Unpaired t-tests were carried out using TIBCO Spotfire™ software. The statistical comparison generated delta delta CT [ddCT] values (the mean dCT of the treated group−the mean dCT of the control group). The statistical software converts the ddCT values into log and linear RQ values for export [RQ=2−ddCT]. The linear RQ values have been converted to linear fold-change values to simplify data interpretation; linear fold-change data was calculated from exported linear RQ values using Microsoft Excel:
For RQ values>1.0: Linear fold-change value=RQ value
For RQ values<1.0: Linear fold-change value=−1/RQ value
   Linear fold-change values of 2.0 or greater are typically considered biologically relevant, but in the personal care industry, fold-change values of 1.5 are often seen in marketing materials.

Sample concentration and purity were determined using a Nanodrop™ spectrophotometer. The A260/280 readings indicate sample purity with ideal measurements that range from 1.8-2.1. The A260/230 ratio is an additional measure of sample purity where a value of 1.0 or greater is ideal. As shown in Table 4, each sample yielded a sufficient quantity of high purity RNA for Standard Skin Panel processing. RNA yields were high, with similar yields among all three groups.

TABLE 4
RNA Sample Quantity and Quality

| | | RNA Isolation | | | Vacuum Concentration | | |
|---|---|---|---|---|---|---|---|
| Genemarkers | | ng/μL | | | ng/μL | | |
| Sample ID | Treatment Group | RNA | 260/280 | 260/230 | RNA | 260/280 | 260/230 |
| A19-001 | 2% NL20181002 | 348.4 | 2.13 | 2.15 | | | |
| A19-002 | 2% NL20181002 | 240.9 | 2.12 | 2.18 | | | |
| A19-003 | 2% NL20181002 | 294.1 | 2.13 | 2.19 | | | |
| A19-004 | 2% NL20181002 | 218.0 | 2.13 | 2.20 | | | |
| A19-005 | 2% NL20181004 | 283.2 | 2.13 | 2.20 | | | |
| A19-006 | 2% NL20181004 | 309.9 | 2.13 | 2.20 | | | |
| A19-007 | 2% NL20181004 | 164.3 | 2.12 | 2.15 | 212.5 | 2.14 | 2.15 |
| A19-008 | 2% NL20181004 | 232.7 | 2.12 | 2.20 | | | |
| A19-009 | Vehicle | 294.4 | 2.13 | 2.17 | | | |
| A19-010 | Vehicle | 396.2 | 2.13 | 2.19 | | | |
| A19-011 | Vehicle | 193.4 | 2.13 | 2.20 | 243.8 | 2.14 | 2.15 |
| A19-012 | Vehicle | 246.3 | 2.13 | 2.21 | | | |

An endogenous control gene that is consistently expressed in all of the samples of a comparison was selected. Three algorithms were used to calculate stability scores and determine the most consistent endogenous control gene: Normfinder algorithm, Minimum Variance Median algorithm, and calculating the coefficient of variability (CV). Lower stability scores represent more consistent expression between samples in the study. Five candidate control genes were analyzed (GAPDH, GUSB, HPRTI, PPIA, and UBC); and substantial uniformity of CT values across all 24 samples was seen.

Table 5 shows the average ranking was calculated across all three algorithms. Based on the stability scores and average rankings, PPIA was the most stable gene. PPIA was selected as the most stable endogenous control gene, and statistics (unpaired t-tests) were carried out using dCT values normalized to PPIA.

TABLE 5
Endogenous Control Gene Selection, Algorithm Ranks

| | | | | | Mean |
|---|---|---|---|---|---|
| Gene | normfinder | minvarmed | CV | ranks | Rank |
| GAPDH | 9.32E−02 | 7.59E−02 | 2.51E−02 | 5, 5, 5 | 5.000 |
| GUSB | 8.32E−02 | 5.73E−03 | 1.11E−02 | 3, 4, 1 | 2.667 |
| HPRT1 | 8.68E−02 | 1.62E−04 | 1.37E−02 | 4, 1, 3 | 2.667 |
| PPIA | 5.40E−02 | 1.18E−03 | 1.18E−02 | 1, 2, 2 | 1.667 |
| UBC | 7.13E−02 | 2.23E−03 | 1.83E−02 | 2, 3, 4 | 3.000 |

Bold text is used to indicate the two lowest stability scores for each algorithm, and the lowest mean rank.

### Example 4: Clinical Evaluation of Effect on Skin

The objective of this study was to clinically evaluate the ability of test products to elicit an improvement in wrinkles, elasticity/firmness and hydration of the skin in women over 40 years of age. This was a single-center monadic split-face design study over a 4-week period. Subjects tested two facial skin moisturizer test articles. Efficacy of the test articles was assessed using analysis of visual assessments, Cutometer and Trans Epidermal Water Loss (TEWL) measurements, and photography using a professional camera (Visia-CR) at baseline, week 2, and week 4. Instrument readings and photographs were also taken 1 hour following the first test article application on Day 1. Thirteen female subjects completed the study summarized in table 6.

TABLE 6
Test results for 13 female subjects using the facial serums

| HTR Code | Description | Treatment |
|---|---|---|
| A-1002 | Facial serum containing active | Applied twice per day |
| A-1003 | Facial serum containing active | Applied twice per day |
| A-1004 | Facial serum containing active | Applied twice per day |
| D | Facial serum | Applied twice per day |

There were no adverse events reported during the course of the study. Subjects were assigned one of the three test articles (A-1002, 1003, and -1004) and the placebo (HTR Code D). Each of the three test articles corresponds to peptide samples 1002, 1003 and 1004 respectively. Four to five subjects completed the study for each test article and the paired placebo (HTR Code D).

### Clinical Evaluations of Fine Lines, Coarse Wrinkles, and Dryness (Baseline, Days 15 and 30)

Review of the data showed larger decreases (improvement) from baseline by Day 30 in fine line scores for sites treated with A-1002, A-1003, and A-1004 than the facial serum (HTR Code D). Changes from baseline for sites assigned Codes A-1003 and A-1004 also showed decreases in evaluations of coarse wrinkles by Day 30. Changes from baseline for dryness for the test article treated sites were minimal or equivalent to placebo sites by Day 30. Statistical analyses found no statistically significant changes from baseline or significant differences between the test article and placebo treated sites for fine lines, coarse wrinkles, and dryness at any time point.

### Cutometer R6 and R7 (Baseline, 1-Hour Post Application on Day 1, and Days 15 and 30): R6 (Visco-Elasticity) Readings

Code A-1003 sites exhibited the smallest change from baseline 1 hour post treatment on Day 1. Codes A-1002 and A-1004 showed the smallest changes from baseline on Days 15 and 30. Statistical analyses found no statistically significant changes from baseline. In one instance, a statistically significant difference was found between sites where Code A-1004 sites increased from baseline compared to a decrease from baseline for the facial serum (HTR Code D) sites.

### R7 (Biological Elasticity) Readings

Only Code A-1004 sites showed an increase (improvement) from baseline 1 hour post treatment on Day 1. Sites treated with Codes A-1003 and A-1004 sites exhibited smaller decreases from baseline than sites treated with Code A-1002 on Days 15 and 30. Statistical analyses of the data found significant decreases from baseline in R7 values on Day 15 for both Code A-1002 and the facial serum (Code D). There were no statistically significant differences between the test article and placebo treated sites at any time point.

### Transepidermal Water Loss (TEWL) Readings (Baseline, 1-Hour Post Application on Day 1, and Days 15 and 30)

Review of the data found greater improvement (decreases) in TEWL by Day 30 for the treated sites than placebo sites. A statistically significant change from baseline was found in one instance only, Day 15, where the placebo sites paired with Code A-1003 showed a significant decrease in TEWL from baseline. Statistical analyses found no statistically significant differences between the test article and placebo treated sites for TEWL.

### Example 4—Clinical Evaluation Over 8-Weeks

The objective of this study was to clinically evaluate the ability of test products to elicit an improvement in wrinkles, elasticity/firmness and hydration of the skin in Caucasian women between 40-60 years of age. This was a single-center monadic split-face design study over an 8-week period. Each subject tested one of two facial skin moisturizer test articles on one side of the face and the placebo on the other side (per randomization). Efficacy of the test articles was assessed using analysis of visual assessments, Cutometer and Trans Epidermal Water Loss (TEWL) measurements, and photography using a professional camera (Visia-CR) at Baseline on Day 1 and Weeks 2, 4, 6, and 8. Instrument readings and photographs were also taken 1 hour following the first test article application on Day 1.

Forty female subjects completed the study. A total of 19 subjects completed the study for Code A and its paired serum (Code D) and 21 subjects completed the study for Code B and its paired serum (Code D).

The test articles were facial serums identified as follows. In each case of A and B, the hemp peptides were spray-dried powder from peptide sample 1002, described above:

Code D (Standard Reference Serum)

| | | |
|---|---|---|
| Ingredient Name | Phase | % |

| | |
|---|---|
| Water | 96.400% |
| Solagum AX | 0.500% |
| Hyaluronic Acid | 0.100% |
| Aloe Vera 200X | 0.100% |
| Euxyl PE 9010 | 0.800% |
| Gypsy Fragrance | 0.100% |
| Water | 2.000% |

Code A

| Ingredient Name | Phase | % |
|---|---|---|
| Propanediol | | 7.000% |
| DL-Panthenol | A | 2.000% |
| Aloe Vera Conc 200X | PM1 | 0.150% |
| Caffeine (anhydrous) | HEAT | 1.000% |
| Niacinamide | | 1.000% |
| Marine Pancogene 2 | | 2.000% |
| Gatuline Intense | | 2.500% |
| Gatuline Expressions | | 3.000% |
| Gatuline Fresh Cells Kiwi | | 3.000% |
| Symrise Sym3D | | 0.100% |
| DHEA | | 1.000% |
| Solagum AX | | 0.600% |
| Hyaluronic Acid | | 0.500% |
| Water | B | 63.350% |
| Vitamin C water soluble | A in order | 1.000% |
| Euxyl PE 9010 | | 0.800% |
| Hydresia SF-2 | C | 8.000% |
| Vitamin A | | 0.200% |
| Hemp Peptide | | 2.000% |
| Fragrance | | 0.300% |
| Vitamin E | PM2 | 0.500% |
| INGREDIENTS | | 100.000% |

Code B

| Ingredient Name | Phase | % |
|---|---|---|
| Water | | 77.350% |
| Aloe vera | | 0.100% |
| Propanediol | A | 3.000% |
| Solagum AX | | 0.500% |
| Glycolic Acid | | 0.000% |
| Lactic Acid | | 0.000% |
| Salicylic Acid | | 0.000% |
| Allantoin | | 1.000% |
| Skinmimics | | 1.000% |
| Marine Pancogene 2 | | 2.000% |
| CCT | | 1.500% |
| Hydresia SF2 | | 5.000% |
| Hyaluronic acid | | 0.500% |
| Euxyl PE 9010 | | 0.800% |
| Canadian Willowherb | | 2.000% |
| Nelupure | B | 2.000% |
| Vitamin E | | 0.500% |
| Vitamin A | | 0.500% |
| Hemp Peptide | | 2.000% |
| Fragrance | | 0.200% |
| Ph adjuster | | 0.050% |
| | | 100.000% |

Subjects were assigned one of two test articles (Codes A or B) and the serum (Code D). An initial conditioning phase was two days in duration. During this time, subjects were asked to discontinue use of all eye creams, facial moisturizers and treatment products except for the conditioning product provided, a brand name deep clean facial cleanser, through the duration of the conditioning phase. Make-up was permitted as long as it did not contain moisturizing ingredients. During this time, subjects were instructed to wash their face with provided cleanser and to avoid prolonged exposure to the sun. Subjects were provided with a non-moisturizing SPF 50 to wear if outdoors for longer than 30 minutes.

Baseline Visit (Day 1)—Study enrollment began on the first day of the study. Potential subjects arrived at the lab with the face free of facial make-up. If subjects had make-up on their face, they were asked to remove all make-up and/or cosmetics on their face and then sit for 30 minutes prior to instrumental measurements and photography. Subjects were screened for entrance onto the study through completion of a medical history screener form and examination by the visual evaluator (including identification of the worse side of the face, based on the score for fine lines). Qualified subjects, with a minimum score of 4 for fine lines, were assigned a final subject identification number. Upon entrance into the study, subjects completed at least 15 minutes of acclimation in an environmentally controlled room maintained with conditions of 19.5° C. to 21.7° C. and 30% to 37% relative humidity, prior to instrumental measurements and photography on each cheek.

Following baseline photographs and assessments, the test article was dispensed. Test article application instructions were given verbally and in written form and the first test article application was made at the lab under supervision. One hour (±20 minutes) after the application, subjects completed a 15-minute acclimation in a temperature and humidity controlled room followed by photographs and instrumental readings to measure elasticity, firmness and skin hydration.

Treatment Period (Day 2 — 59)—The treatment period consisted of an additional 58 days of twice-daily applications of the test article to the face (total of 59 days of application with Baseline application at the test center and all remaining applications unsupervised). Subjects were instructed to apply the test article twice daily to the assigned side, per randomization. Subjects were instructed to avoid direct contact with the eyes and not to use or try any new or different products on their face. Subjects were also instructed to bring all test articles to each visit (Weeks 2, 4, 6, and 8) at the test center. During the treatment period, subjects continued use of Neutrogena facial cleanser soap for all general facial cleansing and if needed, subjects were provided with a non-moisturizing SPF 50 to wear, if outdoors for longer than 30 minutes. Subjects were instructed to avoid use of any additional products on the face aside from what was provided (other than the usual non-moisturizing make-up they use). On evaluation days, subjects were asked to report to the test site with a clean face free of make-up. If appointments were in the morning, subjects were asked not to apply test product until after their study visit. Eye and lip make-up were permitted, however no jewelry. Subjects were instructed not to wet their face within three hours of a study visit. During the study, subjects documented application of the test article and any comments regarding the test article on a diary form. The last application was on Day 59, there was no application at the final visit (Day 60).

Weeks 2, 4, 6, and 8 (Days 15, 30, 45, and 60)—Subjects arrived at the test center with a clean face free of make-up. If appointments were in the morning, subjects were asked not to apply test product until after the study visit. Eye and lip make-up were permitted, however no jewelry. Subjects completed a 15-minute acclimation in a temperature and humidity controlled room and had visual evaluations, instrumental readings to measure elasticity, firmness and skin hydration and photographs (1 image was collected per cheek; total of 2 images per subject) using a professional camera (Visia-CR).

On Days 15, 30, and 45 a compliance check, review of the diaries, and an adverse event (AE) query were performed by a qualified technician. All used and unused test articles were weighed.

On Day 60, a compliance check was done, and the test articles and diaries were returned to the test center. A final AE query was conducted by a qualified technician. Subjects were permitted to keep the conditioning soap. All used and unused test articles were collected and weighed.

### Instrument Evaluations

Instrument assessments were performed with a TEWL RG1 and Cutometer dual MPA580. Subjects acclimated for 15 minutes in a temperature and humidity controlled room maintained at 18.9° C. to 22.3° C. and 23% to 39% relative humidity prior to any instrument readings.

An overview of the results of statistical analyses is provided below.

### Clinical Evaluations of Fine Lines, Coarse Wrinkles, and Dryness at Baseline and Days 15, 30, 45, and 60

Analysis of the changes from baseline found statistically significant improvement in fine lines from Day 30 through Day 60 for both CR Code A and the paired serum sites (CR Code D) shown in FIG. 10. Statistically significant improvement in fine lines was also found for CR Code B (FIG. 11) and the paired serum sites (CR Code D) on Days 45 and 60.

Statistical analyses found no statistically significant differences between the test articles (CR Codes A or B) and their paired serum (CR Code D) for fine lines, coarse wrinkles, or dryness at any time point shown in FIGS. 12 and 13.

TABLE 1A
Within Treatment Analysis for Fine Lines

| CR | | | Mean change | Standard | Signed Rank |
|---|---|---|---|---|---|
| Code | Visit | N | from Baseline | Deviation | Test p-value |
| A | Day 15 | 20 | −0.300 | 1.7502 | >0.5000 |
| D | Day 15 | 20 | −0.100 | 1.5183 | >0.5000 |
| A | Day 30 | 20 | −1.100 | 1.5183 | 0.0098¹ |
| D | Day 30 | 20 | −0.800 | 1.0052 | 0.0078¹ |
| A | Day 45 | 20 | −1.900 | 1.5183 | 0.0002¹ |
| D | Day 45 | 20 | −2.000 | 1.2978 | <0.0001¹ |
| A | Day 60 | 19 | −2.105 | 1.2425 | <0.0001¹ |
| D | Day 60 | 19 | −2.421 | 1.4266 | <0.0001¹ |
| B | Day 15 | 21 | −0.286 | 1.5856 | >0.5000 |
| D | Day 15 | 21 | −0.286 | 1.9272 | >0.5000 |
| B | Day 30 | 21 | −0.762 | 1.6095 | 0.0762 |
| D | Day 30 | 21 | −0.762 | 1.4800 | 0.0574 |
| B | Day 45 | 21 | −1.905 | 1.3381 | <0.0001¹ |
| D | Day 45 | 21 | −2.000 | 1.4142 | <0.0001¹ |
| B | Day 60 | 21 | −2.000 | 1.7889 | 0.0004¹ |
| D | Day 60 | 21 | −2.667 | 1.7127 | <0.0001¹ |

¹Indicates statistically significant decrease compared to baseline.

TABLE 2A
Within Treatment Analysis for Coarse Wrinkles

| CR | | | Mean change | Standard | Signed Rank |
|---|---|---|---|---|---|
| Code | Visit | N | from Baseline | Deviation | Test p-value |
| A | Day 15 | 20 | 0.200 | 1.4364 | >0.5000 |
| D | Day 15 | 20 | 0.100 | 1.2096 | >0.5000 |
| A | Day 30 | 20 | −0.100 | 1.3727 | >0.5000 |
| D | Day 30 | 20 | −0.200 | 1.2814 | >0.5000 |
| A | Day 45 | 20 | −0.300 | 1.7502 | >0.5000 |
| D | Day 45 | 20 | −0.400 | 1.3917 | 0.3438 |
| A | Day 60 | 19 | −0.421 | 1.5747 | 0.3877 |
| D | Day 60 | 19 | −0.526 | 1.3068 | 0.1797 |
| B | Day 15 | 21 | 0.667 | 0.9661 | 0.0156¹ |
| D | Day 15 | 21 | 0.381 | 1.2032 | 0.2891 |
| B | Day 30 | 21 | 0.095 | 0.7684 | >0.5000 |
| D | Day 30 | 21 | 0.190 | 1.7782 | >0.5000 |
| B | Day 45 | 21 | 0.095 | 1.1792 | >0.5000 |
| D | Day 45 | 21 | −0.381 | 1.4992 | 0.3877 |
| B | Day 60 | 21 | 0.000 | 1.4142 | >0.5000 |
| D | Day 60 | 21 | −0.190 | 1.4007 | >0.5000 |

¹Indicates statistically significant increase compared to baseline.

TABLE 3A
Within Treatment Analysis for Dryness

| CR | | | Mean change | Standard | Signed Rank |
|---|---|---|---|---|---|
| Code | Visit | N | from Baseline | Deviation | Test p-value |
| A | Day 15 | 20 | −0.100 | 0.3078 | 0.5000 |
| D | Day 15 | 20 | 0.050 | 0.2236 | >0.5000 |
| A | Day 30 | 20 | 0.050 | 0.5104 | >0.5000 |
| D | Day 30 | 20 | 0.050 | 0.2236 | >0.5000 |
| A | Day 45 | 20 | 0.000 | 0.4588 | >0.5000 |
| D | Day 45 | 20 | 0.150 | 0.3663 | 0.2500 |
| A | Day 60 | 19 | 0.211 | 0.4189 | 0.1250 |
| D | Day 60 | 19 | 0.211 | 0.4189 | 0.1250 |
| B | Day 15 | 21 | 0.000 | 0.0000 | — |
| D | Day 15 | 21 | 0.000 | 0.0000 | — |
| B | Day 30 | 21 | 0.000 | 0.0000 | — |
| D | Day 30 | 21 | 0.000 | 0.0000 | — |
| B | Day 45 | 21 | 0.238 | 0.4364 | 0.0625 |
| D | Day 45 | 21 | 0.190 | 0.4024 | 0.1250 |
| B | Day 60 | 21 | 0.190 | 0.4024 | 0.1250 |
| D | Day 60 | 21 | 0.095 | 0.3008 | 0.5000 |

Note:
p-value missing due to all value are zero at Day 15 and Day 30 for CR code B and D.

TABLE 4A
Within Treatment Analysis for Cutometer
Measurements (R6 Reading)

| CR | | | Mean change | Standard | T- Test |
|---|---|---|---|---|---|
| Code | Visit | N | from Baseline | Deviation | p-value |
| A | 1-1 Hour | 21 | 0.258 | 0.2188 | <0.0001¹ |
| D | 1-1 Hour | 21 | 0.173 | 0.1605 | <0.0001¹ |
| A | Day 15 | 20 | 0.136 | 0.0634 | <0.0001¹ |
| D | Day 15 | 20 | 0.093 | 0.0974 | 0.0004¹ |
| A | Day 30 | 20 | 0.111 | 0.1281 | 0.0010¹ |
| D | Day 30 | 20 | 0.083 | 0.0816 | 0.0002¹ |
| A | Day 45 | 20 | 0.077 | 0.1124 | 0.0066¹ |
| D | Day 45 | 20 | 0.054 | 0.1118 | 0.0454¹ |
| A | Day 60 | 19 | 0.063 | 0.1446 | 0.0746 |
| D | Day 60 | 19 | 0.063 | 0.1626 | 0.1089 |
| B | 1-1 Hour | 22 | 0.261 | 0.1835 | <0.0001¹ |
| D | 1-1 Hour | 22 | 0.229 | 0.1707 | <0.0001¹ |
| B | Day 15 | 21 | 0.156 | 0.1142 | <0.0001¹ |
| D | Day 15 | 21 | 0.156 | 0.1062 | <0.0001¹ |
| B | Day 30 | 21 | 0.098 | 0.1185 | 0.0012¹ |
| D | Day 30 | 21 | 0.121 | 0.1397 | 0.0008¹ |
| B | Day 45 | 21 | 0.056 | 0.1008 | 0.0202¹ |
| D | Day 45 | 21 | 0.047 | 0.1128 | 0.0719 |
| B | Day 60 | 21 | 0.112 | 0.1218 | 0.0004¹ |
| D | Day 60 | 21 | 0.080 | 0.1424 | 0.0186¹ |

¹Indicates statistically significant increase compared to baseline.

TABLE 5A
Within-Treatment Analysis for Cutometer
Measurements (R7 Reading)

| CR | | | Mean change | Standard | T- Test |
|---|---|---|---|---|---|
| Code | Visit | N | from Baseline | Deviation | p-value |
| A | 1-1 Hour | 21 | 0.122 | 0.0737 | <0.0001¹ |
| D | 1-1 Hour | 21 | 0.109 | 0.1002 | <0.0001¹ |
| A | Day 15 | 20 | 0.020 | 0.0617 | 0.1599 |
| D | Day 15 | 20 | 0.008 | 0.0778 | >0.5000 |
| A | Day 30 | 20 | 0.027 | 0.0479 | 0.0190¹ |
| D | Day 30 | 20 | 0.008 | 0.0633 | >0.5000 |
| A | Day 45 | 20 | 0.033 | 0.0471 | 0.0055¹ |
| D | Day 45 | 20 | 0.003 | 0.0672 | >0.5000 |
| A | Day 60 | 19 | 0.008 | 0.0448 | 0.4611 |
| D | Day 60 | 19 | 0.004 | 0.0675 | >0.5000 |
| B | 1-1 Hour | 22 | 0.107 | 0.0773 | <0.0001¹ |
| D | 1-1 Hour | 22 | 0.101 | 0.0871 | <0.0001¹ |
| B | Day 15 | 21 | 0.022 | 0.0530 | 0.0684 |
| D | Day 15 | 21 | 0.025 | 0.0589 | 0.0645 |
| B | Day 30 | 21 | 0.024 | 0.0508 | 0.0447¹ |
| D | Day 30 | 21 | 0.048 | 0.0654 | 0.0033¹ |
| B | Day 45 | 21 | 0.015 | 0.0392 | 0.0952 |
| D | Day 45 | 21 | 0.022 | 0.0737 | 0.1855 |
| B | Day 60 | 21 | 0.007 | 0.0460 | 0.4732 |
| D | Day 60 | 21 | 0.005 | 0.0479 | >0.5000 |

¹Indicates statistically significant increase compared to baseline.

TABLE 6A
Within-Treatment Analysis for TEWL measurements

| CR | | | Mean change | Standard | T- Test |
|---|---|---|---|---|---|
| Code | Visit | N | from Baseline | Deviation | p-value |
| A | 1-1 Hour | 21 | 0.001 | 2.1841 | >0.5000 |
| D | 1-1 Hour | 21 | 0.231 | 2.0382 | >0.5000 |
| A | Day 15 | 20 | 3.938 | 4.4336 | 0.0008² |
| D | Day 15 | 20 | 6.041 | 7.0289 | 0.0011² |
| A | Day 30 | 20 | 4.300 | 4.4876 | 0.0004² |
| D | Day 30 | 20 | 5.344 | 6.4958 | 0.0016² |
| A | Day 45 | 20 | 2.038 | 4.0936 | 0.0383² |
| D | Day 45 | 20 | 1.769 | 3.5913 | 0.0402² |
| A | Day 60 | 19 | 2.940 | 3.1742 | 0.0008² |
| D | Day 60 | 19 | 2.845 | 3.5006 | 0.0023² |
| B | 1-1 Hour | 22 | −1.080 | 1.6103 | 0.0049¹ |
| D | 1-1 Hour | 22 | −0.295 | 1.3017 | 0.3006 |
| B | Day 15 | 21 | 3.701 | 5.5832 | 0.0065² |
| D | Day 15 | 21 | 4.370 | 4.6295 | 0.0003² |
| B | Day 30 | 21 | 2.370 | 3.7045 | 0.0082² |
| D | Day 30 | 21 | 3.379 | 3.2251 | 0.0001² |
| B | Day 45 | 21 | 0.253 | 3.1007 | >0.5000 |
| D | Day 45 | 21 | 1.194 | 4.3147 | 0.2192 |
| B | Day 60 | 21 | 1.291 | 1.9656 | 0.0069² |
| D | Day 60 | 21 | 2.666 | 2.6236 | 0.0002² |

¹Indicates statistically significant decrease compared to baseline.
²Indicates statistically significant increase compared to baseline.

### Cutometer R6 and R7 (Baseline, 1-Hour on Day 1, and Days 15, 30, 45, and 60)

R6 (Visco-elasticity) Readings: Review of the changes from baseline found the largest increase 1-hour post application for the test articles (CR Codes A and B) and serum sites (CR Code D); generally these increases tended to subside over the eight week use period. Analysis of the changes from baseline found statistically significant increases at each time point through Day 45 for CR Codes A and D (FIG. 15). Statistically significant increases from baseline were also found at each time point for CR Codes B and D with the exception of Day 45 for CR Code D sites (FIG. 16). Between-treatment analyses found no significant differences between the test articles and their paired serum sites with the exception of one time point, Day 15, for CR Codes A and D sites (CR Code A sites showed a larger increase from baseline than Code D sites.)

R7 (Biological Elasticity) Readings: Review of the changes from baseline found the largest increase 1-hour post application on Day 1 for the test articles and control. Analysis of the changes from baseline found statistically significant increases from baseline at 1-hour on Day 1 and Days 30 and 45 for CR Code A sites and at 1-hour only for its paired serum (CR Code D) shown in FIG. 17. Statistically significant increases from baseline were also found for both CR Code B and its paired serum (CR Code D) at 1-Hour and Day 30 (FIG. 18). Between-treatment analyses found no significant differences between the test articles and their paired control sites with the exception of one time point, Day 45, for CR Codes A and D sites (CR Code A sites showed a larger increase from baseline than Code D sites.)

Transepidermal Water Loss (TEWL) Readings at Baseline, 1-hour on Day 1 and Days 15, 30, 45, and 60: CR Code A sites and its paired serum (CR Code D) showed statistically significant increases from baseline in TEWL from Days 15 through Day 60 (FIG. 19). CR Code B sites exhibited a significant decrease in TEWL I-hour post application on Day 1, followed by significant increases in TEWL for both CR Codes B and D on Days 15, 30, and 60 (FIG. 20). Statistical analyses found no statistically significant differences in TEWL between the test articles (CR Codes A or B) and their paired control sites.

### Example 5: Nutraceutical Beverage Formulations

The following flavoured beverage compositions were prepared:

| Ingredient | Batch 1 | 2 | 3 | 4 |
|---|---|---|---|---|
| water | 97.3419 | 97.3499 | 97.3375 | 97.0670 |
| fruit powder/flavor* | 0.7500 | 0.76 | 0.61 | 0.82 |
| citric acid | 0.2000 | 0.25 | 0.35 | 0.400 |
| stevia extract | 0.0330 | 0.04 | 0.0225 | 0.0330 |
| guar gum | 0.0200 | 0.025 | 0.025 | 0.025 |
| fish collagen peptides | 1.0673 | 1.0672 | 1.0672 | 1.0672 |
| hemp peptide | 0.5000 | 0.5000 | 0.5000 | 0.5000 |
| hyaluronic acid | 0.0411 | 0.0411 | 0.0411 | 0.0411 |
| ascorbic acid | 0.0241 | 0.0241 | 0.0241 | 0.0241 |
| Vitamin D3 | 0.0022 | 0.0022 | 0.0022 | 0.0022 |
| Vitamin E | 0.0090 | 0.0090 | 0.0090 | 0.0090 |
| B3 Niacin | 0.0056 | 0.0056 | 0.0056 | 0.0056 |
| B5 Pantothenic Acid | 0.0020 | 0.0020 | 0.0020 | 0.0020 |
| biotin | 0.0038 | 0.0038 | 0.0038 | 0.0038 |
| TOTAL | 100 | 100 | 100 | 100 |

*Different combinations of grapefruit powder, orange powder, pomegranate powder, cranberry powder, acai powder, coconut water powder, mango powder, passionfruit powder, lime powder, grapefruit flavour, coconut flavour, mango flavour, etc.

### REFERENCES

The following references are provided to indicate the relative level of skill in the art, and are incorporated by reference in their entirety, where permitted.
Das, R. (2015) Multienzyme modification of hemp protein for functional peptides synthesis. J. Food Proc. 2015: 1-5.

### DEFINITIONS AND INTERPRETATION

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated. To the extent that the following description is of a specific embodiment or a particular use of the invention, it is intended to be illustrative only, and not limiting of the claimed invention.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to combine, affect or connect such aspect, feature, structure, or characteristic with other embodiments, whether or not such connection or combination is explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited, and such terms refer to ranges that can be subsequently broken down into sub-ranges as discussed above. In the same manner, all ratios recited herein also include all sub-ratios falling within the broader ratio.

1. A skin and/or hair care composition for topical use to prevent, mitigate or treat a skin condition or a hair condition in a subject, wherein the composition comprises a mixture of peptides produced by enzymatic hydrolysis of hemp proteins, the peptide mixture comprising low-molecular weight hemp (LMHW) peptides which pass through a 3 kDa ultrafiltration filter.

2. The composition of claim 1 which further comprises a dermatologically acceptable carrier.

3. The composition of claim 1 wherein the peptide mixture comprises or consists essentially of peptides less than about 1 kDa.

4. The composition of claim 1 wherein the peptide concentration is between 0.05 and 20.0% by weight, preferably between 0.1 and 5.0%, and most preferably between 0.5 and 2.0%.

5. The composition of claim 1, wherein the enzymatic hydrolysis is done by one or both of a serine endopeptidase and a fungal protease.

6. The composition of claim 1 wherein the LMWH peptides comprise at least one or more of the following peptides:

| Peptide Amino | |
|---|---|
| Acid Sequence | SEQ ID NO. |
| PETF | 1 |
| ASPR | 2 |
| WS | |
| PSR | |
| RPS | |
| MAR | |
| ARSQ | 3 |
| NDNA | 4 |
| SRDN | 5 |
| IQNT | 6 |
| NQA | |
| YNL | |
| LKY | |
| YNL | |
| LIAF | 7 |
| RARS | 8 |
| LRQN | 9 |
| VTRSGA | 10 |
| HAC | |
| LQNRDI | 11 |
| PGCPET | 12 |
| VSEYW | 13 |
| VSEYW | 14 |
| ERGTSS | 15 |

7. The composition of claim 1 which is a cleanser, toner, serum, cream, lotion or a mask.

8. A method of preventing, mitigating or treating a skin condition or a hair condition in a subject, comprising the step of topically applying a composition of claim 1.

9. (canceled)

10. The method of claim 8 wherein the peptides are produced by enzymatic hydrolysis of hemp proteins.

11. The method of claim 10, wherein the peptides comprises one or more of the following peptides:

| Peptide Amino | |
|---|---|
| Acid Sequence | SEQ ID NO. |
| PETF | 1 |
| ASPR | 2 |
| WS | |
| PSR | |
| RPS | |
| MAR | |
| ARSQ | 3 |
| NDNA | 4 |
| SRDN | 5 |
| IQNT | 6 |
| NQA | |
| YNL | |
| LKY | |
| YNL | |
| LIAF | 7 |
| RARS | 8 |
| LRQN | 9 |
| VTRSGA | 10 |
| HAC | |
| LQNRDI | 11 |
| PGCPET | 12 |
| VSEYW | 13 |
| VSEYW | 14 |
| ERGTSS | 15 |

12. The composition of claim 5, wherein the serine endopeptidase comprises subtilisin A.

13. The composition of claim 5 wherein the fungal protease comprises Flavourzyme.
